(19) United States
(12) Patent Application Publication
(10) Pub. No.: US 2003/0023181 A1
(43) Pub. Date: Jan. 30, 2003

(54) Gas analyzer of the fluorescent-film type particularly useful for respiratory analysis

(76) Inventor: James R. Mault, Evergreen, CO (US)

Correspondence Address: Allen M. Krass, Gilford, Krass, Groh, Sprinkle, Anderson & Citkowski, P.C., 280 N. Old Woodward Ave., Suite 400, Birmingham, MI, 48009-5394, US

(21) Appl. No.: 10/202,745

(22) Filed: Jul. 25, 2002

Related U.S. Application Data
(60) Provisional application No. 60/308,043, filed on Jul. 26, 2001.

(51) Int. Cl. A61B 5/08

(52) U.S. Cl. 600/532

(57) ABSTRACT

An analyzer for detecting at least one gas component of a gas, particularly useful for analyzing a respiratory gas, includes a flow channel for conducting the gas; a removable liner removably lining the inner surface of the flow channel, the removable liner carrying a fluorescent film having at least one fluorescent element effective, when excited by an excitation source, to emit fluorescent radiation of a particular frequency quenchable by a gas component of the gas; a detector for detecting the fluorescent radiation; and an excitation source for exciting the fluorescent film to emit the fluorescent radiation. In one embodiment, the removable liner is transparent; and in a second embodiment, a transparent member is used for carrying the fluorescent film.

### REFERENCE TO RELATED APPLICATIONS

The present application includes subject matter described in Provisional Application Serial No. 60/308,043, filed Jul. 26, 2001, which application is hereby incorporated by reference, and claims the priority date of that application. The present application is also related to patent application Ser. No. 10/162,371, filed Jun. 4, 2002, titled Respiratory Analyzer And Method For Measuring Changes In Concentration Of A Gas Component Of A Breathing Gas Mixture, which application also includes subject matter of Provisional Application Serial No. 60/308,043.

### FIELD OF THE INVENTION

The present invention relates to analyzers for detecting at least one gas component of a gas mixture. The invention is particularly useful in respiratory analyzers for analyzing respiratory gases during inhalations and exhalations thereof, and is therefore described below with respect to that application.

### BACKGROUND OF THE INVENTION

A respiratory analyzer includes a flow channel through which a subject breathes a breathing gas mixture, typically atmospheric air, and one or more sensors which sense and measure one or more gas components of the breathing gas mixture. Such analyzers are widely used for measuring metabolism and related respiratory parameters, by indirect calorimetry in a diet or weight-control program.

The term “respiratory analyzer”, as used herein, refers to any device used to study the breath of a person, such as an indirect calorimeter (including oxygen consumption meters and carbon dioxide production meters), breath diagnostic systems such as breath alcohol meters, ventilator control systems, spirometers, nitric oxide meters, end tidal analyzers, systems for cardiac output determination, and other devices. A particularly important application of such analyzers is to measure oxygen concentration, which may be done in a variety of ways. The preferred embodiments of the invention described below utilize oxygen sensors of the fluorescent-type, where molecular oxygen is the quenching species.

Accordingly, the invention is described below particularly with respect to such sensors, but it will be appreciated that the invention can be used with gas component sensors other than the fluorescent-type.

The fluorescent-type sensor provides a fluorescence signal correlated with the partial pressure of the gas component to be sensed (e.g., oxygen) in the respired gases. Consider, for example, inhaled gas having an inhaled oxygen partial pressure Pᵢ, and exhaled gas having an exhaled oxygen partial pressure Pₑ. It will also be assumed that the fluorescent signal is related to a fluorescent intensity of a sensing channel, although fluorescence decay and attack times can also be used to determine oxygen partial pressures in a manner known to the art.

The fluorescence sensor provides a fluorescent intensity signal Fᵢ during inhalation, and a fluorescent intensity signal Fₑ during exhalation. The difference in fluorescence signals (ΔF), which equals Fₑ−Fᵢ is correlated with the difference in oxygen partial pressures (ΔP), which equals Pᵢ−Pₑ.

An oxygen sensor can be calibrated so that Fₑ and fᵢ, have a known correlation with Pₑ and Pᵢ, using a numerical or analytical relationship or some combination thereof. The term “fluorescence-oxygen relationship” will be used to refer to the fluorescence intensity/oxygen partial pressure relationship and the fluorescence decay time (or excited state lifetime)/oxygen partial pressure relationship for a fluorescent material. The term “sensor response function” will be used in a more general sense, to refer to a fluorescence-oxygen relationship, but also to other sensor responses to analytes. The fluorescence-oxygen relationship determined for a new sensor (i.e. before degradation) will be termed the “initial fluorescence-oxygen relationship”.

U.S. Pat. Nos. 5,917,605, 5,910,661, 5,894,351, and 5,517,313, the contents of which are incorporated herein by reference, describe fluorescence sensors for various analytes, including oxygen and glucose in a fluid. An oxygen sensing film can include, for example, a ruthenium (II) complex irradiated by an excitation radiation source so as to induce an orange-red fluorescence. The fluorescence is quenched by the presence of molecular oxygen, reducing the fluorescence intensity in a manner which can be correlated with the partial pressure of oxygen.

Luminescent decay time sensors are described by Wolfbeis et al. in Sensors and Actuators B51, 17-24 (1998), incorporated herein by reference.

Sensors can include a sensing channel and a reference channel. The sensing channel can be, for example, a fluorescent film exposed to the fluid and providing a fluorescence signal correlated with the partial pressure of the fluid component of interest. The reference channel can be a similar fluorescent film exposed to the same environmental conditions, such as temperature and excitation radiation intensity, but not exposed to the fluid. A fluorescence intensity ratio (between the sensing channel fluorescence intensity and the reference channel fluorescence intensity) will be correlated with the partial pressure of the fluid component, but not correlated with environmental factors common to both channels, such as the instantaneous temperatures or excitation radiation intensity. The use of a reference channel therefore provides compensation for the instantaneous temperature and/or other environmental influences.

Fluorescence quenching and fluorescence lifetime of fluorescence quenching sensors follow the Stern-Vollmer equation, for example, as discussed in U.S. Pat. No. 6,074,607 to Slovacek et al. and U.S. Pat. No. 5,518,694 to Bentsen, the contents of which are incorporated herein by reference. The equation is written in the form:

F₀/F=τ₀/τ=1+k_{q}τ₀[Q]

where F₀ is the fluorescence intensity for zero concentration of the quencher, F is the fluorescence intensity in the presence of the quencher, τ₀ is the excited state lifetime for zero concentration of the quencher, τ is the excited state lifetime in presence of the quencher, k_{q} is the bimolecular fluorescence quenching rate constant, and [Q] is the concentration of the quencher in the fluorescent sensor film.

This equation is often written as:

F/F₀=τ₀/τ=1+K_{SV}P_{Q}

where k_{SV} is the Stem-Vollmer coefficient, and P_{Q} is the partial pressure of the quencher.

Molecular oxygen (O₂) is known to quench the fluorescence of various fluorescent films. The Stern-Vollmer coefficients account for the solubility of oxygen, or other analyte, in the fluorescent element. In the description below, the representation P is used to refer to the partial pressure of analytes, such as oxygen or carbon dioxide in fluid flow.

Fluorescence decay times, or excited state lifetimes, can be measured using excitation source modulation techniques, for example as described by U.S. Pat. No. 5,518,694 to Bentsen. Fluorescent intensities are conventionally measured using a photodetector receiving the fluorescence. An optical filter can be used to prevent excitation radiation from reaching the photodetector.

U.S. patent application Ser. No. 09/630,398 describes an indirect calorimeter, particularly useful for determining metabolic rates in a dietary management and/or weight control program, including a flow pathway, a flow sensor, and an oxygen sensor. The integration of flow rate and oxygen partial pressure gives oxygen volumes in respired gases. The oxygen consumed by a person is the difference between inhaled and exhaled oxygen volumes (corrected to standard conditions). A metabolic rate may be determined from the consumed oxygen volume. In other embodiments, a carbon dioxide sensor or capnometer can be used instead of or in addition to the oxygen sensor to determine metabolic rate.

U.S. patent application Ser. No. 09/630,398 also describes an oxygen sensor having a sensing channel and a reference channel. The sensing channel includes a fluorescent film exposed to changes in oxygen partial pressure in respired gases passing through the indirect calorimeter, and provides a fluorescence signal correlated with the oxygen partial pressure in the respired gases. The reference channel is part of the same sensor package and is similarly affected by ambient conditions such as temperature. However, the reference channel is not exposed to the oxygen in the respired gas. Therefore, the ratio of the sensing channel fluorescence signal to the reference channel fluorescence signal is correlated with oxygen partial pressure in the respired gases, but not with ambient conditions, as the effects of ambient conditions on sensing and reference channels (ideally) cancel out. The use of a reference channel, however, adds to the cost and complexity of the sensor.

In embodiments of an indirect calorimeter described in U.S. patent application Ser. No. 09/630,398, a single-point recalibration technique is used to compensate for the temperature changes of the oxygen sensor. Single point recalibration of an oxygen sensor is also described in the above-cited Bentsen U.S. Pat. No. 5,518,694.

There are many other applications where it is desired to sense two or more components of a gas mixture. For example, it may be desirable to sense change in both the oxygen concentration, and also the carbon dioxide concentration, in a respiratory gas following inhalation and exhalation.

Such fluorescent-type sensors are rapidly degradable over a period of time, and therefore require frequent recalibration and/or replacement. The above-cited co-pending patent application Ser. No. 10/162,371, filed Jun. 6, 2002, relates to a technique for compensating for degradation of the fluorescent film, thereby substantially reducing the need for frequent recalibration and/or replacement. The present application relates to a construction of such an analyzer which enables replacement of the fluorescent film to be effected in a simple, quick and efficient manner.

The following documents are incorporated herein by reference: Provisional Application Serial No. 60/308,043, filed Jul. 26, 2001, patent application Ser. No. 10/162,371, filed Jun. 4, 2002.

### SUMMARY OF THE PRESENT INVENTION

According to one aspect of the present invention, there is provided an analyzer for detecting at least one gas component of a gas, comprising a flow channel for conducting the respiratory gas; a removable liner removably lining the inner surface of the flow channel, the removable liner carrying a fluorescent film having at least one fluorescent element effective, when excited by an excitation source, to emit fluorescent radiation of a particular frequency quenchable by a gas component of the gas; and an excitation source for exciting the fluorescent film to emit the fluorescent radiation.

It will thus be seen that since the degradable fluorescent film is carried by a removable liner, the fluorescent film may be replaced in a quick and convenient manner by merely removing the old liner from the flow channel, and substituting a new liner with a fresh fluorescent film.

According to further features in preferred embodiments of the invention described below, the fluorescent film includes at least two fluorescent elements each quenchable by different gas component of the gas. In some described preferred embodiments, the two fluorescent elements are defined by different fluorescent regions of the fluorescent film. In other described embodiments, the two fluorescent elements are defined by different chemical compounds in the same region of the fluorescent film.

According to another aspect of the present invention, there is provided a gas analyzer for detecting at least one gas component of a gas mixture, comprising a gas analyzer for detecting at least one gas component of a gas mixture, comprising a flow channel for conducting the gas; a transparent member having an inner surface facing said flow channel, and a fluorescent film carried by said inner surface; said fluorescent film having at least one fluorescent element effective when excited by an excitation source to emit fluorescent radiation of a particular frequency quenchable by a gas component of the gas; a detector for detecting the fluorescent radiation; and an excitation source for exciting said fluorescent film to emit the fluorescent radiation.

Further features and advantages of the invention will be apparent from the description below.

### BRIEF DESCRIPTION OF THE DRAWINGS

The invention is herein described, by way of example only, with reference to the accompanying drawings, wherein:

FIG. 1 diagrammatically illustrates the main optical elements of one form of respiratory analyzer constructed in accordance with the present invention;

FIG. 2 is a block diagram illustrating the basic components of the electrical system in the analyzer of FIG. 1;

FIG. 3 illustrates the main optical elements of another respiratory analyzer constructed in accordance with the present invention;

FIGS. 4 and 5 illustrate two further constructions of respiratory analyzers in accordance with the present invention in which two fluorescent elements for detecting two different gas components are defined by different fluorescent regions of a fluorescent film, or by two fluorescent films;

FIGS. 6 and 7 illustrate two further constructions including a single fluorescent film in which the two (or more) fluorescent elements are defined by different chemical components in the same region of the fluorescent film; and

FIG. 8 illustrates one channel of another gas analyzer construction that may be used with a fluorescent film sensing one or more gas components.

### DETAILED DESCRIPTION OF THE PREFERRED EMBODIMENTS

The gas analyzer illustrated in FIGS. 1-8 of the accompanying drawings is particularly useful in an indirect calorimeter, such as described in U.S. patent application Ser. No. 09/630,398, for determining metabolic rates in a dietary management and/or weight control program. Such a calorimeter includes a flow pathway for the respiratory gas, a flow sensor, and an oxygen sensor. By measuring the oxygen concentration, and also the air volume, during inhalation and exhalation, the calculator determines the amount of oxygen consumed, and thereby the metabolic rate of the individual. Other embodiments are described in which a carbon dioxide sensor can be used instead of, or in addition to, the oxygen sensor, also to provide a measurement of metabolic rate, or to provide other information useful in a dietary management and/or weight-control programs.

Further details of the construction and operation of such a respiratory analyzer, or indirect calorimeter, are available from the above-cited patent application Ser. No. 09/630,398, the contents of which are incorporated herein by reference.

Sensors according to the present invention can also be used in respiratory analysis, in conjunction with flow sensors, to determine ventilatory equivalent, respiratory quotient, end tidal concentrations, and cardiac output. Sensors according to the present invention can be used to detect other respiratory components, such as

The fluorescent sensor illustrated in FIG. 1 is carried by a removable liner, generally designated 2, receivable within a flow tube 4 defining a flow channel 6 for conducting the respiratory gas to be analyzed. Removable liner 2, or a part thereof, is of a transparent material. Its inner surface carries two fluorescent sensor films 8, 10, respectively, so as to be contacted by the respiratory gas flowing through channel 6. For example, one film could be oxygen-sensitive, and the other film could be carbon dioxide sensitive. Alternatively, both films could be oxygen sensitive, with one film serving as a reference for the other. In such cases, the film serving as a reference would be shielded from the gas mixture in the flow channel so as to be subject to the same temperature and other ambient conditions of the sensing film, except that it would not be contacted by the gas, and therefore could serve as a reference for compensating for ambient conditions.

As shown in FIG. 1, the flow tube 4 receiving the removable liner 2, carries two detectors 12, 14, each in radial alignment with one of the fluorescent films 8, 10. Flow tube 4 further carries a single excitation source 16 located between the two detectors, 12, 14. Since at least the portion of liner 2 carrying the sensor films 8, 10 is of transparent material, the single excitation source 16 is effective to excite both fluorescent films 8, 10.

The two fluorescent films 8, 10 for sensing the gas components of interest being carried by the removable transparent liner 2, may thus be conveniently replaced by merely replacing the liner.

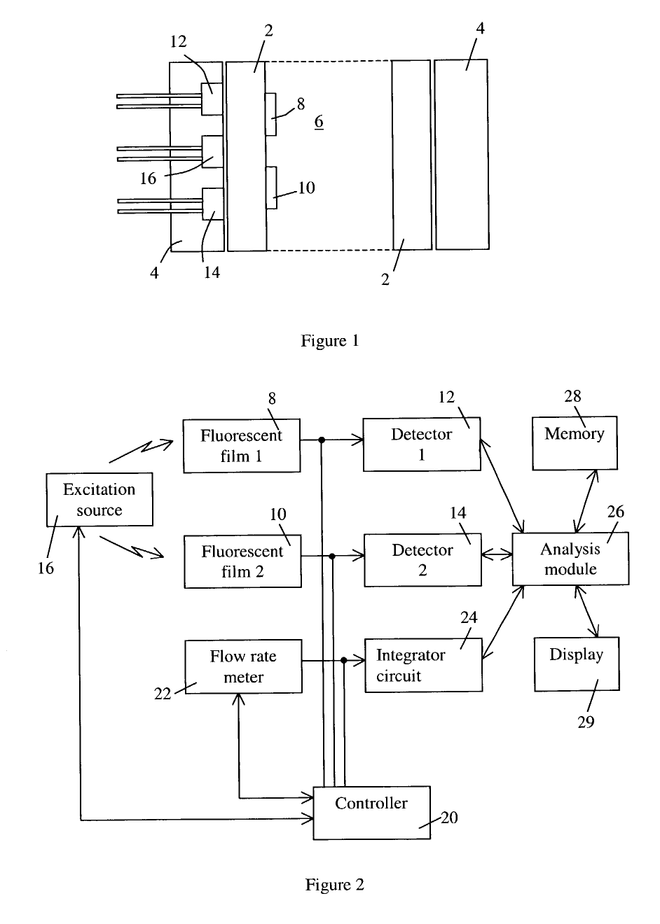

FIG. 2 schematically illustrates the electrical circuit for operating and controlling the foregoing elements of the respiratory analyzer of FIG. 1. As shown in FIG. 2, the excitation source 16 is controlled by a controller 20 to excite the two fluorescent films 8, 10. As indicated earlier, the fluorescent radiation emitted by these films, when excited, are extinguished by the gas components, oxygen and carbon dioxide in this case, to be detected. The fluorescent radiation emitted by the two fluorescent films 8, 10 are thus detected by the respective detectors 12, 14, which produce outputs corresponding to the concentration of the respective component gases in the respiratory stream of gases flowing through the flow channel 6 of FIG. 1.

The system illustrated in FIG. 2 further includes a flow rate sensor 22 which detects the flow direction, and also the flow rate, of the respired gases. The flow rate is integrated by an integrating circuit 24 over a period of time of the respective inhalation and exhalation, to thereby produce an output representing the volume of the gas during the respective inhalation and exhalation.

The concentration outputs of the two detectors 12, 14, and the air volume output of the flow rate sensor 22 and its integrating circuit 24 are fed to an analysis module 26 which analyzes this information in accordance with the method described in the U.S. patent application Ser. No. 09/630,398, to produce measurements of the changes in concentration of the two gas components (oxygen and carbon dioxide) and thereby a measurement of the metabolic rate. The analysis module 26 may also compensate for the degradation of the fluorescent films 8, 10 caused by usage, in the matter described in the above-cited co-pending patent application Ser. No. 10/162,371, filed Jun. 4, 2002

The outputs of the analysis module 26 may be stored in a memory 28, and/or displayed in a display 29.

Further details of the construction and operation of the electrical circuit illustrated in FIG. 2 may be had from U.S. patent application Ser. No. 09/630,398, incorporated herein by reference.

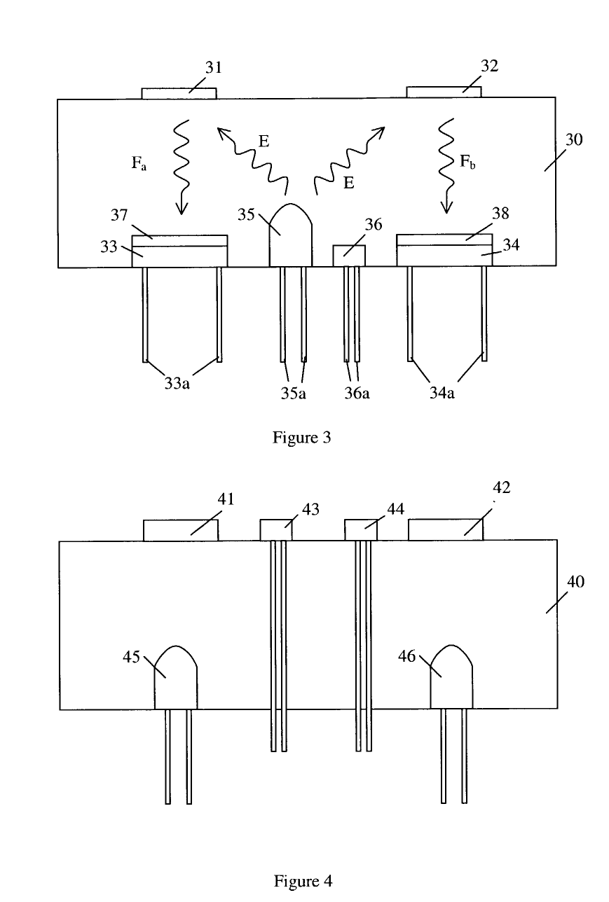

FIG. 3 illustrates another respiratory analyzer constructed in accordance with the present invention, including a transparent member 30 carrying two fluorescent films 31, 32 on a surface to be exposed to the respiratory gas flowing through the flow channel. Transparent member 30 may also be in the form of a transparent, removable liner lining the inner surface of the flow channel as in FIG. 1; alternatively, it could be in the form of a solid block of transparent material inserted within an opening in the flow channel so as to expose the two fluorescent films 31, 32 for contact with the respiratory gas flowing through the flow channel.

Transparent member 30 also carries, on the surface thereof opposite to the two fluorescent films 31, 32, a detector 33, 34, each in alignment with one of the films. The detector side of the transparent member 30 further carries an excitation source 35 located between the two fluorescent films. A heating element 36 adjacent to the excitation source 35 is used to maintain the fluorescent films at a predetermined temperature above ambient, such as a temperature of 39° F., so as to reduce the effects of condensation on the fluorescent films. This also helps reduce temperature variations of the fluorescent films. The excitation source 35 is preferably a light emitting diode (LED) energized by electrical leads 35a. The electrical heater 36 is energized by leads 36a, whereas the outputs of the two detectors 33, 34 appear on their respective output leads, 33a, 34a.

Each of the detectors 33, 34, is provided with a filter 37, 38, for passing to the respective detector only the fluorescent excitation from the respective fluorescent film 31, 32. Thus, as shown in FIG. 3, the excitation radiation from excitation source 35 to the respective fluorescent film 31, 32 is indicated by the two wavy arrows E; whereas the fluorescent radiation emitted by the respective fluorescent films 31, 32 to their respective detectors 33, 34, is indicated by the wavy arrows at Fₐ and F_{b}, respectively.

In one preferred embodiment of respiratory analyzer illustrated in FIG. 3, fluorescent film 31 is a separate film, or a region on a common film, of a fluorescent material, such as ruthenium complex, sensitive to oxygen; whereas fluorescent film 32 would be a separate film, or a region on a common film sensitive to carbon dioxide.

FIG. 4 illustrates another respiratory analyzer, similar to that of FIG. 3, also including a transparent member 40 having a fluorescent film 41 sensitive to one gas component, and a second fluorescent film 42 sensitive to a second gas component, both carried on the surface of the transparent member 40 to be exposed to the flowing respiratory gas. As in the embodiment of FIG. 3, the two fluorescent films 41, 42, could be separate distinct films, or separate regions on the same film.

The fluorescent radiation emitted by each of the fluorescent films 41, 42, is also detected by a separate detector 43, 44 for each film. In this case, however, the two detectors 43, 44 are carried on the same surface of the transparent member 40 as the two fluorescent films 41, 42.

The two fluorescent films 41, 42 are illuminated by two excitation sources 45, 46, embedded within transparent member 40 or carried on its opposite surface, in alignment with the respective fluorescent film 41, 42.

It will thus be seen that in the respiratory analyzer of FIG. 4, the two fluorescent films 41, 42, are excited by their respective excitation sources 45, 46, to emit fluorescent radiation which is detected by their respective detectors 43, 44, to provide a measurement of the concentration of the two gas components of interest in the respiratory gases. If desired, each detector 43, 44, could be provided with a filter to permit passage therethrough only the fluorescent radiation for the respective detector.

The transparent member 40 illustrated in FIG. 4 may also be part of a removable transparent liner lining the flow channel as described above with respect to FIG. 1; alternatively, it could be a separate transparent member which is received within an opening in the flow channel so as to expose the two fluorescent films 41, 42 to the gas flowing through the flow channel.

In other embodiments, the transparent member 40 may comprise two parts, a first part enclosing or covering one or more excitation sources or detectors, and a second part supporting one or more fluorescent films. The first part may be a component of a respiratory analyzer. The second part may be removable, for example as part of a removable flow tube liner. The two parts of the transparent member may have a curved or flat interface, as desired.

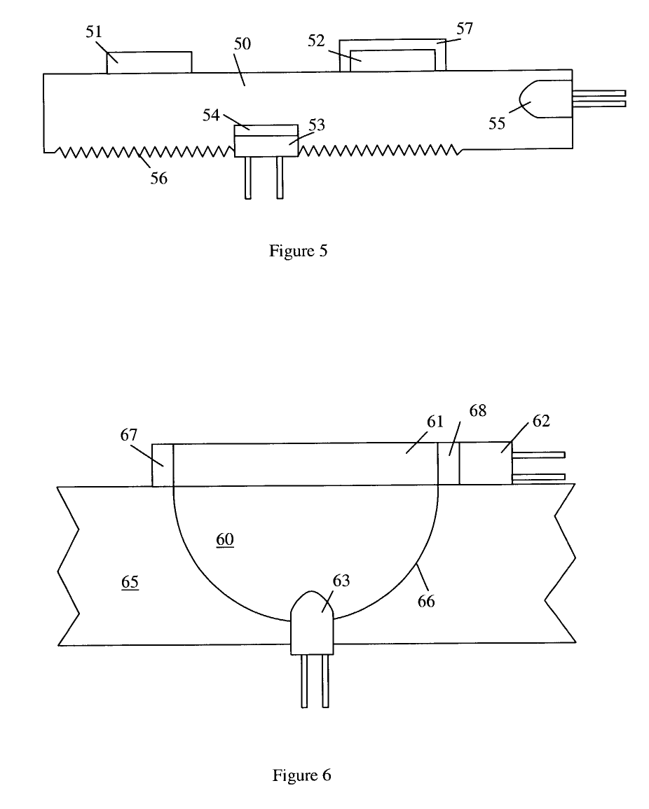

FIG. 5 illustrates another respiratory analyzer including a transparent member 50 carrying a first fluorescent film 51 and a second fluorescent film 52 on one face of the member for exposure to the flowing gas. In this case, there is a single detector 53 and a single excitation source 55. The single detector 53 is located between the two fluorescent films 51, 52, whereas the single excitation source 55 is located laterally of the two fluorescent films and the detector. The surface of transparent member 50 opposite to that carrying the two fluorescent films 51, 52, is roughened, as shown at 56, to reflect the excitation radiation from source 55 towards the two fluorescent films 51, 52.

It will thus be seen that the excitation radiation from source 55 undergoes diffused reflection from the rough surface 56 so as to excite the two fluorescent films 51, 52, to emit fluorescent radiation. The fluorescent radiation is detected by detector 53. A filter 54 may be provided to pass only the respective fluorescent radiation.

The analyzer illustrated in FIG. 5 could be used in applications providing a first fluorescent film serving as a sensor for the gas component of interest, and a second fluorescent film serving as a reference for compensating for temperature or other ambient conditions. In such an application, the sensor fluorescent film 51 would be directly exposed to the gas component of interest, whereas the fluorescent film 52 would be shielded by a gas-impermeable shield, schematically shown at 57, from the gas flowing through the flow channel.

Where a single detector is provided, as shown at 53 in FIG. 5, the output channel of the detector 53 could be selectively enabled so as to receive the fluorescent emissions from the two fluorescent films 51, 52 at different time intervals, thereby enabling the output of the detector to distinguish between the fluorescent emissions from the two films 51,52.

As in the earlier-described embodiments, the transparent member 50 (or a part of it supporting fluorescent films) could be a transparent replaceable liner, or a part thereof, or could be a transparent member received within an opening in the flow channel. In addition, the two fluorescent films 51, 52 could be separate and distinct films, or could be separate regions on the same film.

In the above-described embodiments, the fluorescent film includes two (or more) fluorescent elements, in the form of distinct films or distinct regions on a common film, each of which fluorescent elements is quenchable by a different gas component of the respiratory gas (or by the same gas component in the case of a reference sensor). The two (or more) fluorescent elements could also be defined by different chemical compounds in the same region of the fluorescent film. For example, a fluorescent film can include a first fluorescent compound (such as a first transition metal complex) having a fluorescence quenched by oxygen, and a second fluorescent compound (such as a second transition metal complex) having a fluorescence quenched by carbon dioxide. Using a single detector, the response of the fluorescent film to different modulation frequencies can be used to determine the partial pressure of oxygen and carbon dioxide from the time dependence of the two fluorescence decays within the film. The detector signal phase response to at least two modulation frequencies can be determined. If a single excitation source is used, two modulation frequencies can be used in a time-sequential manner, or a single modulation waveform having at least two frequency components can be used. Alternatively, using a single modulation frequency of the excitation source, the detector signal can be analyzed, filtered, or otherwise processed so as to determine two phase components corresponding to the time-dependent response of the first and second fluorescent compounds.

FIG. 6 illustrates a gas analyzer including a transparent member 60 carrying a single fluorescent film 61, a single detector 62, and a single excitation source 63. In this case, the transparent member 60 is mounted on a mounting member 65 and includes a reflecting layer at the interface 66 between the transparent member and the mounting member for directing the excitation radiation from source 63 towards the fluorescent film 61. The fluorescent film 61 is on the face of the transparent member 60 exposed to the gas being analyzed. That face is circumscribed by another reflector layer 67, which extends completely around the fluorescent film 61 except for a window in alignment with the detector 62. A filter 68 is provided in this window to pass to the detector only the fluorescent radiation emitted by the fluorescent film 61 when excited by the excitation source 63.

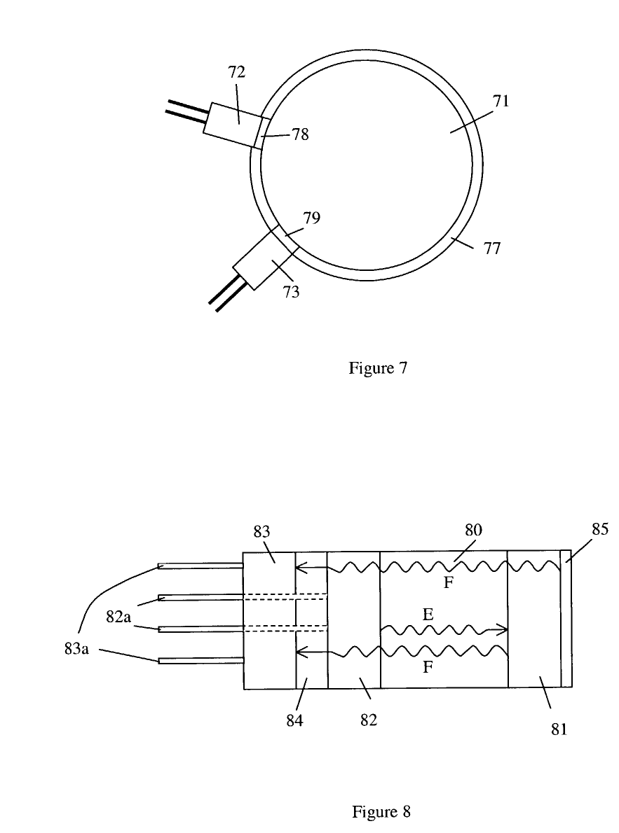

FIG. 7 is a top view of a gas analyzer having a construction similar to that of FIG. 6, including a single fluorescent sensor film 71 overlying a transparent member (corresponding to transparent member 60 in FIG. 6), and having a single detector 72 laterally of the fluorescent film 71. In this case, however, the excitation source 73 is also laterally of the fluorescent film 71. A reflector layer 77 completely circumscribes the fluorescent film 71 except for a first window 78 in alignment with the detector 72, and a second window 79 in alignment with the excitation source 73.

In this example, the fluorescent film can act as a waveguide to guide fluorescence to the detector. The refractive index of the transparent member 60, or other supporting substrate, can be chosen to be less than the refractive index of the fluorescent film so as to enhance the waveguiding effect.

In other embodiments, the film 71 may be irradiated by an excitation source out of the plane of the fluorescent film (for example, as shown in FIG. 6), and two (or more) detectors disposed around the side of the fluorescent film, for example at locations 72 and 73 in FIG. 7. The fluorescent film may be plane, or may be curved, for example to match the curvature of a flow tube.

FIG. 8 illustrates another arrangement which may be used as a single-channel sensor for sensing one particular gas component, or as one channel of a multi-channel sensor for sensing two or more gas components.

The sensor illustrated in FIG. 8 includes a transparent member 80 having a fluorescent film 81 on one surface for direct exposure to the gas being analyzed, and an excitation source 82 on the opposite surface for exciting the fluorescent film 81 to emit fluorescent radiation. The sensor further includes a detector 83 for detecting the fluorescent radiation emitted by the fluorescent film 81. The sensor also includes a filter 84 between the excitation source 82 and detector 83 for blocking from the detector the excitation radiation from excitation source 82 (illustrated by wavy arrow E), and for passing only the fluorescent radiation (indicated by wavy arrow F) emitted by the fluorescent film 81. The excitation source 82 is energized via input leads 82a. The output from the detector 83 appears on the output leads 83a.

The sensor illustrated in FIG. 8 further includes a dichroic mirror layer 85 overlying the fluorescent film 81. Dichroic mirror 85 is effective to pass through it the excitation radiation, and to reflect back to the detector 83 the fluorescent radiation. The dichroic mirror layer 85 would be porous or otherwise made permeable to the gases in the gas being analyzed to enable such gases to directly contact the fluorescent film 81.

The dichroic mirror layer 85 may also be in the form of a thin interferometric film, or may be omitted. Other gas permeable mirrors, such as a metallic mesh or porous film, may be used in its place. The detector and radiation source may be a unitary semiconductor device, fabricated by epitaxial processes well known in the art.

It will be appreciated that the transparent member 80 in the embodiment of FIG. 8, as well as the transparent member in the earlier described embodiments, may be in the form of a removable liner, or part of such liner, applied to the inner surface of a flow channel for conducting the gas being analyzed, as described above with respect to FIG. 1; alternatively, transparent member 80 may be a separate member received within an opening in the flow channel.

In all embodiments, the wavelength of excitation radiation can be lower than the optimum wavelength for maximum fluorescence intensity so as to reduce photodegradation effects. For example, a green LED or blue-green LED can be used in place of a blue LED to excite ruthenium complexes. The excitation radiation frequency can be chosen to be the minimum to excite any fluorescence signal. A wavelength-dependent figure of merit, such as sensor lifetime multiplied by fluorescence efficiency and by detector efficiency, may be calculated and optimized. Two photon absorption and upconversion effects can be used to reduce the excitation frequency required.

A fluorescent film can comprise two or more fluorescent species sensitive to different gas components, fluorescence species sensitive to primarily to temperature (i.e. not having a fluorescence sensitive to the presence of respiratory components, or being shielded from respiratory components) and other components to extend the life of the sensor film, such as components providing a deexcitation pathway for excited gas molecules. For example, a first fluorescent species (such as a first transition metal complex) may be included in a film, sensitive to oxygen; a second fluorescent species (such as a second transition metal complex) may be included, sensitive to carbon dioxide; and a third component may be included to provide a non-radiative de-excitation route for singlet oxygen. The fluorescent species or other components can be dispersed within a matrix, or supported upon a support layer, such as may comprise a polymer, glass, solgel, ormosil, array of columns, mesh, network or other structure permeable to the gas component(s) of interest. Microspheres containing or supporting the fluorescent species can be enclosed, partially sintered, or otherwise held together or supported on the substrate.

Further, a fluorescent sensor can comprise a fluorescent species having a fluorescence sensitive to a partial pressure of a first gas component, and a second component (either within a fluorescent film or within a separate calorimetric film) providing a colorimetric response indicating the presence of a second gas component. For example, a fluorescent oxygen sensor can further comprise a qualitative calorimetric indicator of second respiration component, such as acetone, other ketones, nitric oxide, or other diagnostic breath component. The colorimetric response can be determined by absorption, reflection, or transmission of radiation, for example at the same wavelength as may used to excite a fluorescence-type sensor film, or at a different wavelength.

A single detector can be located so as to receive fluorescence from a plurality of fluorescent species. For example, in some embodiments, fluorescence from two or more regions, each sensitive to a different respiratory component (for example, one species sensitive to oxygen and another species sensitive to carbon dioxide) can reach the same detector. The detection scheme may use polarization switching, modulation, phase lag detection, switchable optical filters, optical shutters, beam steering devices, or other techniques to determine the fluorescent parameters of more than one fluorescent species.

A single fluorescent film (or other fluorescent region) may comprise a plurality of fluorescent species, each species responsive to a different respiratory component. In other embodiments, multiple fluorescent regions may be provided, each sensitive to a one of a number of respiratory components of interest.

The decay time dependence of the fluorescence from a film, or films, comprising two fluorescent species can be written in the form:

F(t)=A₁exp(t1/τ₁)+A₂exp(t/τ₂)

For example, the first fluorescent species (and therefore A₁ and τ₁) may be responsive to oxygen, and the second fluorescent species (and therefore A₂ and τ₂) may be responsive to carbon dioxide. Various techniques can be used to determine the fluorescence parameters (amplitude and time dependent parameters) associated with a single species. Either the amplitude term A, decay time τ, or both A and τ, may be determined. If analysis of the decay time dependence is used, the decay parameters can be determined by mathematical analysis (such as nonlinear least square methods), photon counting techniques, or other methods. The detector response signal phase lag for a plurality of modulation frequencies can also be used to extract a plurality of decay parameters, and this can be facilitated by selecting fluorescent species having decay times which are significantly different from each other, for example, differing by more than an order of magnitude. For example, two modulation frequencies (e.g. of approximately 1/τ₁ and 1/τ₂) can be used to determine two decay times τ₁ and τ₂, from determination of the detector signal phase lag at the two frequencies. Fluorescent intensity can also be determined from time dependent analysis of a fluorescent signal.

A fluorescent signal received by a detector may comprise a contribution from one (or more) fluorescent species sensitive to a respiratory gas component (for example, oxygen), and a second fluorescent species isolated or insensitive to the respiratory gas component, and hence primarily sensitive to temperature. Determination of the decay parameters present in the signal allows simultaneous determination of temperature and, in this example, oxygen concentration. The temperature is found from the decay parameters determined for the second species, and the oxygen concentration is found using the determined temperature and the decay parameters for the first species. For example, an oxygen sensor may comprise a first region containing an oxygen sensitive fluorescent species, having a first fluorescent parameter correlated with oxygen concentration and with temperature, the first region exposed to a flow of gas; a second region containing a fluorescent species not sensitive to oxygen providing a fluorescent signal having a second fluorescent parameter correlated with temperature, an excitation radiation source illuminating both regions, a detector detecting fluorescence from both regions, and circuitry operable to modulate the excitation radiation source, determine the first and second parameters, determine the temperature from the second fluorescent parameter, and to determine the oxygen concentration from the first fluorescent parameter and the determined temperature. The two regions may be separate films, co-mingled fluorescent species in a single film, comprise a plurality of sub-regions, or be otherwise disposed. The fluorescent parameter may comprise decay time, excited state lifetime, intensity, or some other parameter or combination of parameters.

Data collected during inhalation can be used to calibrate the fluorescent-type sensors, if the inhaled gas composition is known. The fluorescent intensity measured under exposure to atmospheric gas can be used to determine the Stern-Volmer parameters of the sensor. A sensor may also be calibrated by passing a flow of dry nitrogen or other calibration gas through the flow tube. The calibration gas can be maintained at a fixed temperature.

The oxygen sensitive fluorescent species is responsive to the presence of oxygen and temperature, and preferably not to other respiratory gas components. Similarly, the carbon dioxide sensitive fluorescent species is preferably not responsive to oxygen. However, depending on the choice of material used in the sensor, the carbon dioxide sensor may be sensitive to the oxygen concentration. In this case, during exhalation, the oxygen concentration can determined from the oxygen sensor signal, and then carbon dioxide concentration can be determined from the determined oxygen concentration and the carbon dioxide sensor signal. Calibration can still occur during an inhalation. The response of the carbon dioxide sensitive fluorescent species is modeled in terms of oxygen concentration and carbon dioxide concentration, and a calibration can occur during inhalation by comparing the actual fluorescent response with that predicted using the model, knowing the inhaled gas concentration.

A simplified sensor response function can be used, chosen so as to be accurate only over concentration ranges found in respired gases.

In other embodiments, a fluorescent species sensitive substantially to temperature, and not significantly sensitive to respiratory components, can be disposed within a fluorescent film as part of a gas sensor, such as an oxygen sensor. The fluorescent parameter determined from this temperature-dependent species can be used to determine the temperature of the film. Oxygen concentration is then determined from determined temperature and the fluorescent parameters of an oxygen sensitive fluorescent species. This allows temperature control of the sensor film to be less closely controlled, or omitted. During an inhalation, the oxygen sensor can calibrated using the determined temperature of the inhaled gas and the determined fluorescence of the oxygen sensitive species, the latter fluorescence which can be then compared with a temperature-dependent model, equation, look-up table, or other calibration process used.

One or more optical fibers can be used to guide excitation radiation from an excitation source to the outer surface of a disposable liner, from where the excitation radiation is transmitted by the liner to a fluorescent film. One or more optical fibers can also be used to guide fluorescent radiation to one or more detector.

A flow tube liner may comprise one or more separable parts, wherein only one part is transparent. A liner may have a transparent or other light transmissive region proximate to the fluorescent films, but be otherwise opaque or poorly transmissive of light.

Respiratory component which may be detected using sensors according to the present invention include one or more of the following: oxygen, carbon dioxide, nitric oxide, organic compounds (such as volatile organic compounds, including ketones (such as acetone), aldehydes (such as acetaldehyde), alkanes (such as ethane and pentane)), nitrogen containing compounds such as ammonia, and sulfur containing compounds (such as hydrogen sulfide), and hydrogen. Respiratory analysis may include detection of respiratory components diagnostic of health (such lung cancer, for example as disclosed by Phillips in U.S. Pat. No. 6,312,390, incorporated herein by reference), metabolism disorders, oral bacteria, other bacteria, asthma and other respiratory tract inflammations, and infections. Respiratory analysis can include detection of respiratory components administered to a person, such as radio-labeled components, anesthetics, sedatives, drugs, and the like, or products of labeled compounds.

Fluorescent regions other than films can be used in embodiments of the present invention, including three-dimensional structures such as coated networks. If decay time analysis is used, the liner on which the fluorescent films are disposed can have a light-transmissive region allowing fluorescence to reach at least one detector, but this region need not be transparent.

The fluorescent films may be disposed on an adhesive strip comprising a transparent polymer film supporting a fluorescent film, and an adhesive film, which may be placed within a marked or shaped region on the inside surface of a disposable liner so as to be proximate to one or more excitation sources or detectors. Decay time analysis may be preferred if an adhesive layer is used. Alternatively, the fluorescent films may be supported on an insert, such as a polymer film, to be placed on or into a removable liner, for example into a slot or other mechanical guide.

Flow sensors which may be used in conjunction with gas or other fluid sensors according to the present invention include: ultrasonic sensors (e.g. using the transit times of ultrasonic pulses having a component of direction parallel to the flow path, sing-around sensor systems, and ultrasonic Doppler sensors detecting frequency changes in ultrasound as it propagates through a gas), differential pressure sensors (such as a pneumotach), turbines, hot wire anemometers and other thermal methods, and vortex shedding sensors (e.g. detecting vortices shed by an element in the flow path).

It will thus be appreciated that the above-described examples of the invention are set forth merely for illustrative purposes, and that many other variations, modifications and applications of the invention may be made.

What is claimed is:

1. An analyzer for detecting at least one gas component of a gas, comprising:
a flow channel for conducting the respiratory gas;
a removable liner removably lining the inner surface of said flow channel, said removable liner carrying a fluorescent film having at least one fluorescent element effective, when excited by an excitation source, to emit fluorescent radiation of a particular frequency quenchable by a gas component of the respiratory gas;
a detector for detecting the fluorescent radiation;
and an excitation source for exciting said fluorescent film to emit said fluorescent radiation.

2. The analyzer according to claim 1, wherein the portion of said removable liner carrying said fluorescent film is transparent, and said detector and excitation source are carried by said flow channel.

3. The analyzer according to claim 1, wherein said excitation source is a light emitting diode (LED).

4. The analyzer according to claim 1, wherein said fluorescent film includes at least two fluorescent elements each quenchable by a different gas component of the gas.

5. The analyzer according to claim 4, wherein said two fluorescent elements are defined by different fluorescent regions of the fluorescent film.

6. The analyzer according to claim 5, wherein said flow channel carries a detector for each of said fluorescent regions.

7. The analyzer according to claim 6, wherein said excitation source is carried by said flow channel between the two detectors.

8. The analyzer according to claim 6, wherein there is an excitation source for each of said fluorescent regions.

9. The analyzer according to claim 4, wherein said two fluorescent elements are defined by different fluorescent chemical compounds in the same region of the fluorescent film.

10. The analyzer according to claim 9, wherein there is a common detector for all said fluorescent elements, said excitation source exciting said fluorescent elements at different frequencies to enable said common detector to distinguish between the different gas components acting to quench said fluorescent elements.

11. The analyzer according to claim 1, wherein said removable liner is transparent, and said fluorescent film is carried on the inner surface of said removable liner for direct contact with the gas flowing through said flow channel.

12. The analyzer according to claim 11, wherein said inner surface of the removable liner also carries a gas-permeable dichroic layer over said fluorescent film; said dichroic layer being effective to pass the excitation radiation there through, and to reflect the fluorescent radiation to said detector.

13. The analyzer according to claim 11, wherein said removable liner further includes a filter between said excitation source and said detector effective to block from said detector the excitation radiation and to pass to said detector the fluorescent radiation.

14. The analyzer according to claim 11, wherein said fluorescent film is circumscribed by a reflector surface except for a window aligned with said detector.

15. A gas analyzer for detecting at least one gas component of a gas mixture, comprising:
a flow channel for conducting the gas;
a transparent member having an inner surface facing said flow channel, and a fluorescent film carried by said inner surface; said fluorescent film having at least one fluorescent element effective when excited by an excitation source to emit fluorescent radiation of a particular frequency quenchable by a gas component of the gas;
a detector for detecting said fluorescent radiation;
and an excitation source for exciting said fluorescent film to emit said fluorescent radiation.

16. The analyzer according to claim 15, wherein said detector and said excitation source are also carried by said transparent member.

17. The analyzer according to claim 16, wherein said excitation source is a light emitting diode (LED).

18. The analyzer according to claim 15, wherein said fluorescent film includes at least two fluorescent elements each quenchable by different gas component of the respiratory gas.

19. The analyzer according to claim 18, wherein said two fluorescent elements are defined by different fluorescent regions of the fluorescent film.

20. The analyzer according to claim 19, wherein said transparent member carries a detector for each of said fluorescent regions.

21. The analyzer according to claim 20, wherein said excitation source is carried by said transparent member between the two fluorescent regions.

22. The analyzer according to claim 18, wherein there is an excitation source for each of said fluorescent regions.

23. The analyzer according to claim 18, wherein said two fluorescent elements are defined by different chemical compounds in the same region of the fluorescent film.

24. The analyzer according to claim 23, wherein said transparent member carries a common detector for all said fluorescent elements, said excitation source exciting said fluorescent elements at different frequencies to enable said common detector to distinguish between the different gas components acting to quench said fluorescent elements.

25. The analyzer according to claim 15, wherein said fluorescent film is carried on the inner surface of said transparent member for direct contact with the respiratory gas flowing through said flow channel.

26. The analyzer according to claim 25, wherein said detector is carried on the surface of said transparent member opposite to that carrying said fluorescent film.

27. The analyzer according to claim 26, wherein said inner surface of the transparent member also carries a gas-permeable dichroic layer over said fluorescent film and effective to pass the excitation radiation there through, and to reflect the fluorescent radiation to said detector.

28. The analyzer according to claim 26, wherein said excitation source is also included in said transparent member and is located between said fluorescent film on the inner surface and said detector on the outer surface.

29. The analyzer according to claim 28, wherein said transparent member further includes a filter between said excitation source and said detector effective to block from said detector the excitation radiation and to pass to said detector the fluorescent radiation.

30. The analyzer according to claim 26, wherein said excitation source is carried on the surface of said transparent member opposite to that tearing said fluorescent film.

31. The analyzer according to claim 26, wherein said excitation source is carried by said transparent member at a remote location with respect to said fluorescent film, the surface of said transparent member opposite to that tearing said fluorescent film being roughened to reflect the radiation to said fluorescent film.

32. The analyzer according to claim 15, wherein said detector is also carried on the inner surface of said transparent member laterally of said fluorescent film.

33. The analyzer according to claim 32, wherein said excitation source is carried by said transparent member radially aligned with said fluorescent film.

34. The analyzer according to claim 32, wherein said fluorescent film is circumscribed by a reflector surface except for a window aligned with said detector.

35. The analyzer according to claim 32, wherein said inner surface of the transparent member also carries a filter between said detector and said excitation source.

36. The analyzer according to claim 32, wherein said excitation source is also carried on the inner surface of said transparent member laterally of said fluorescent film, said fluorescent film being circumscribed by a reflector surface except for a first window aligned with said excitation source, and a second window aligned with said detector.
